(19) United States
(12) Patent Application Publication
(10) Pub. No.: US 2004/0151666 A1
(43) Pub. Date: Aug. 5, 2004

(54) Rodent mammary window for intravital microscopy of orthotopic breast cancer and related method

(75) Inventors: Mark W. Dewhirst, Durham, NC (US); Siqing Shan, Durham, NC (US)

Correspondence Address: JENKINS & WILSON, PA, 3100 TOWER BLVD, SUITE 1400, DURHAM, NC, 27707, US

(73) Assignee: Duke University

(21) Appl. No.: 10/354,367

(22) Filed: Jan. 30, 2003

(51) Int. Cl. A61K 49/00

(52) U.S. Cl. 424/9.2

(57) ABSTRACT

A rodent mammary window for intravital microscopy of orthotopic breast cancer and the related method of use in rodents to provide a useful model to investigate the cellular behavior of implanted tumor cells in an orthotopic environment and to observe the earliest events during angiogenesis. The mammary window is surgically secured in the mammary gland of the rodent on top of implanted tumor fragments or cells that have been implanted into the nipple sinus of the mammary gland. Periodically the rodent is secured to a microscopy platform with a restrainer plate mounted on top of the abdomen of the rodent such that a central aperture in the restrainer plate is aligned with the mammary window and selective aspects of tumor growth are observed by intravital microscopy including microvascular function and angiogenesis in the orthotopic site.

### GOVERNMENT INTEREST

This invention was made with Government support under Grant No. 5P50CA68438 awarded by the NCI and Grant No. 2R01CA40355 awarded by the NIH. The Government has certain rights therein.

### FIELD OF THE INVENTION

The present invention relates to windows for tumor microenvironment studies, and more particularly to a rodent mammary window for intravital microscopy of orthotopic breast cancer and the related method of use.

### RELATED ART

Increasing evidence shows that orthotopic and ectopic organ environments differentially influence tumor cell gene expression tumor growth, invasiveness, angiogenesis, metastasis, drug delivery and sensitivity to therapeutic agents in many tumor types. For example, a spontaneous murine mammary carcinoma, SP1, grew more aggressively in the mammary gland than in the subcutis and exhibited a 10-fold lower 50% lethal tumor burden and earlier metastasis. It has been shown that metastatic behavior is enhanced when tumor cells are implanted orthotopically, and it is inferred that full and efficient expression of this phenotype may involve some interactions with local connective tissue matrix. Results such as this suggest that the mammary gland either selects for growth of distinct tumor subpopulation or induces phenotypic changes leading to tumor progression and the generation of metastatic subpopulations.

Orthotopic breast cancer models with rodent syngeneic tumors or human xenografts have been widely used in the studies of hormone dependency, novel metastasis models, effects of growth factors on angiogenesis and tumor growth, and gene therapy. However, orthotopic breast cancer models for intravital microscopy have not been previously investigated. Such models could possibly provide an important tool to study tumor growth and angiogenesis.

Rodent dorsal skin fold window chambers have been widely used in tumor microcirculation studies since the 1940's. Transparent tissue windows with tumor implants at orthotopic sites have been developed for studies of microcirculation and angiogenesis, such as cranial window for brain tumors, and an abdominal window for pancreatic malignancies. It has been shown that morphological and functional characteristics of tumor vasculature, drug transport and neovascularization of the same tumor types transplanted in orthotopic sites were significantly different from their counterparts grown in ectopic window chambers. Applicants believe that an ideal mammary carcinoma model for tumor microenvironmental studies would combine the unique features of tissue window with growth in an orthotopic organ environment, which allows repeated, continuous and non-invasive monitoring of tumor growth and angiogenesis.

For this purpose applicants have developed a novel rodent mammary window of orthotopic breast cancer model for intravital microscopy. With technical refinement applicants have unexpectedly and surprisingly obtained images of tumor vascularization and microcirculation with optical quality comparable to traditional dorsal skinfold window chambers. This method should be applicable to the study of blood flow, vasoactivity, vascular permeability, oxygen and drug transport and angiogenesis. In further studies it should be possible to carry out serial observations of neovascularization and blood flow changes as well as responses to therapeutic intervention.

Applicants' discovery meets a long-felt need for such an apparatus and method, and the details thereof will be described hereinafter.

### SUMMARY OF THE INVENTION

Most models currently used in experimental oncology research are ectopically transplanted syngeneic or xenografted tumors. A growing body of evidence has shown that orthotopic and ectopic organ environments differentially influence the tumor growth, invasiveness, metastasis and sensitivity to therapeutic treatment. The development and growth of mammary neoplasms are greatly influenced by age, parity, hormones and diet. Although orthotopic breast cancer models have been studied previously, these studies have not been applied to intravital microscopy in live animals. Rodent dorsal skin fold window chambers have been widely used in tumor microcirculation studies since 1940's. However, the disadvantage of this model has been discovered to be that tumors are implanted in a subcutaneous site, a quite different organ environment from the mammary gland.

Applicants believe that an ideal mammary carcinoma model for tumor microenvironmental studies would combine the unique features of tissue windows with growth in an orthotopic organ environment. For this purpose, applicants have developed an orthotopic rat breast cancer model with a transparent window for intravital microscopy. Applicants have been able to obtain static and videotaped images of tumor vascularization and microcirculation with optical quality comparable to traditional dorsal skinfold window chamber. This method will be applicable to the study of blood flow, vasaoactivity, vascular permeability, oxygen and drug transport and angiogenesis. With this model applicants will be able to carry out serial observations of neovascularization and blood flow changes as well as responses to therapeutic intervention in living animals.

Therefore it is an object of the present invention to provide for an apparatus and method to study orthotopic breast cancer models by intravital microscopy in live animals.

It is another object of the present invention to provide an apparatus and method for studying orthotopic breast cancer models by intravital microscopy in rodents by using mammary windows sutured over the mammary gland of a rodent into which tumor fragments or cells have been implanted into the nipple sinus.

It is still another object of the present invention to provide an apparatus and method for use of a mammary window for intravital microscopy of orthotopic breast cancer in rodents to provide a useful model to investigate the cellular behavior of implanted tumor cells in an orthotopic environment and to observe the earliest events during angiogenesis.

It is still another object of the present invention to provide a mammary window and restraining plate for use in an orthotopic mammary carcinoma model for tumor microenvironment studies to be conducted on rodents.

Some of the objects of the invention having been set forth hereinabove, other objects and features of the invention would be better understood and appreciated when taken in connection with the drawings described in detail hereinbelow.

### BRIEF DESCRIPTION OF THE DRAWINGS

FIG. 1 is a top plan view of the mammary window of the present invention.

FIG. 2 is a top plan view of the restraining plate and microscopy stage of the present invention.

FIG. 3 is an isometric view of the restraining plate and microscopy stage shown in FIG. 2.

FIG. 4 is an experiment setting showing a BALB/C mouse with R4 mammary window positioned in a restrainer on microscope stage. The lateral tail vein is catheterized with heparinized saline. The wrapped syringe is filled with rhodamine-dextran.

FIG. 4A is a normal mammary gland from a Fischer 344 rat on POD 4 with the nipple and surrounding epidermis removed. The dilated nipple sinus is filled with a keratin plug.

FIG. 4B is a well established R3230Ac tumor four days after tumor fragment implantation. The epithelium of the nipple sinus has been invaded by tumor.

FIG. 4C is a normal mouse mammary gland showing part of the nipple sinus and main lactiferous duct.

FIG. 4D is a 4T1 tumor 15 days after cell injection. No evidence of inflammation on the surface beneath the acrylic disk is seen.

FIG. 5A shows GFP-R3230Ac tumor growth and angiogenesis. About 3000 tumor cells soaked in a small piece of GELFOAM were implanted into the nipple sinus. There was no visible green fluorescence from POD 0 through POD 4 in the implant. The image on day 8 shows a bright green “ring”, indicating active proliferation of tumor cells in the peripheral area around the implant. Some tumor cells are elongated with fibroblast-like shape. Numerous new microvessels have grown into this green zone from surrounding areas. At the tips of these new vessels, there are immature, blood-containing sprouts from newly formed vessels. These sprouts were replaced by perfused vessels on subsequent days, and new sprouts appeared at the tips of new vessels in the inner areas. Tumor vascular density increases as the viable tumor zone expands inwards (A through D).

FIG. 5B shows R3230Ac growth and vaculature after tumor fragment implantation. E. Vasculature on POD4 after iv rhodamine-dextran injection. Abundant dilated new vessels appeared in areas surrounding the implant with a few new vessels invading inward. F and G show the window disk (arrowheads, 15 mm in diameter) with tumor growing beneath it (POD 20). H. Appearance of vasculature in this well established tumor with iv rhodamine-dextran. L Bright field epiillumination of tumor vessels. Bars in E and H, 400 μm.

FIG. 5C shows GFP-4T1 cells in BALB/C mouse mammary window (POD 3). Most tumor cells were elongated and oriented towards a dense newly formed vessel cluster. Bar=200 μm.

FIG. 6 shows the dynamics of tumor angiogenesis of GFP-4T1 tumor in the BALB/C mouse mammary window. About 200 tumor cells were injected into the mammary gland tissue during surgery on day 0. On day 2, some cells disappeared and remaining cells were located near existing vessels, which had become dilated and tortuous. On day 5, a few new vessels appeared near (arrowheads) and within the tumor cell clusters. On day 7, a small tumor formed with perfused vessels, most in peripheral areas. Tumor size increased with more vessels on day 9 and image with rhodamine-dextran iv showed that the most active angiogenesis occurred in the peripheral zone. Tumor size doubled on day 14 compared with day 11 with abundant vessels in the peritumoral area, indicating that angiogenesis at tumor center is always lagging behind the tumor mass expansion. Bright field epiillumination shows hemorrhage-like spots at tumor center (arrowheads) on day 17 (Day 17 A). At higher magnification with fluorescence epiillumination (Day 17 B), vessels are clearer with a green background, and numerous fine new vessels in the peripheral area of this “hemorrhagic” spot (arrowheads) are shown. This phenomenon is similar to the findings in R3230Ac tumors in FIG. 5A upper panel. Day 17C and Day 20 show highly irregular and heterogeneous tumor vasculature in this model. Bars=200 μm.

FIG. 7 shows microhemorrhage and blood containing sprouts precede new vessel formation. A. A green zone of orthotopic GFP-R3230Ac tumor implant near the host tissue (see Materials and Methods) on POD 5, where many new vessels derived from the host tissue are goring centrally. Above the tips of newly formed vessels, there are numerous blood containing sprouts (arrowheads) and some microhemorrhagic appearing spots, which were replaced by perfused vessels on consequent days. B. Higher magnification view of such hemorrhagic spots. C. Similar findings in an orthotopically implanted GFP-4T1 tumor in BALB/C mouse mammary POD7 (C) and POD 9 (D).

### DETAILED DESCRIPTION OF THE INVENTION

### Materials and Methods

### Mammary Window and Restrainer Plate

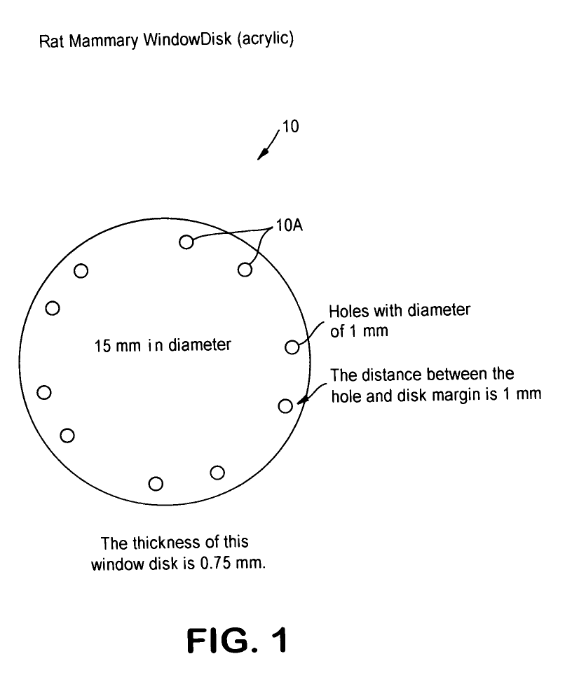

The novel mammary window developed for use in the novel method of the present invention is shown in FIGS. 1-4 and generally designated 10. With reference to FIG. 1, window 10 comprises an acrylic disk about 10-15 mm in diameter (depending upon whether it is sutured to a mouse or a rat, respectively) and has a thickness of about 0.75 mm. Ten (10) apertures or holes 10A defining a diameter of about 1.0 mm are positioned around the perimeter of disk 10 to facilitate suturing to the rodent.

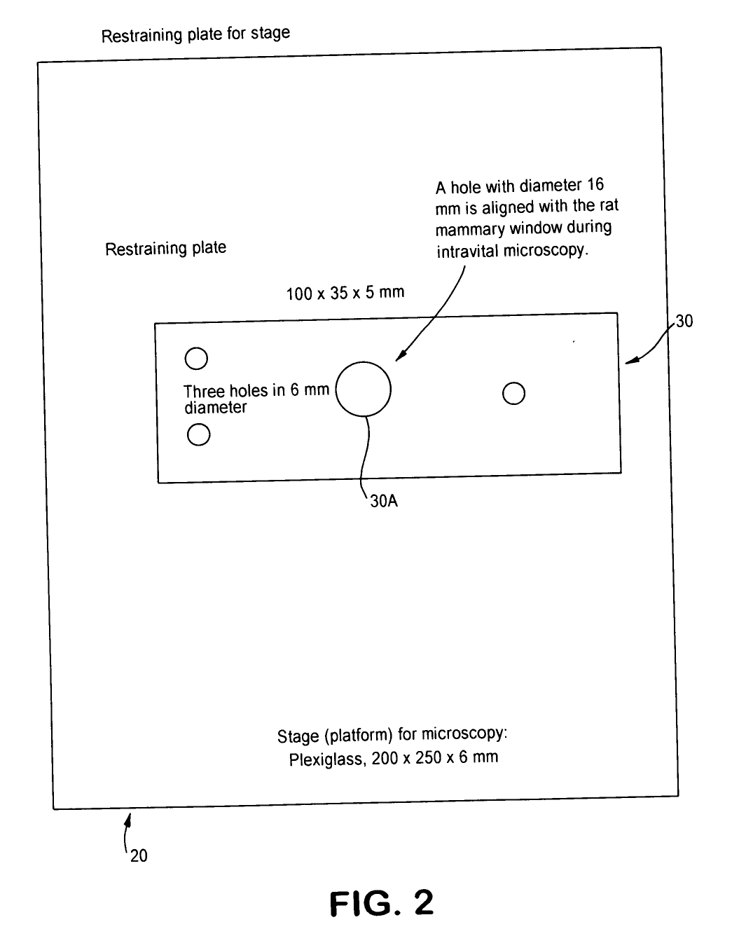

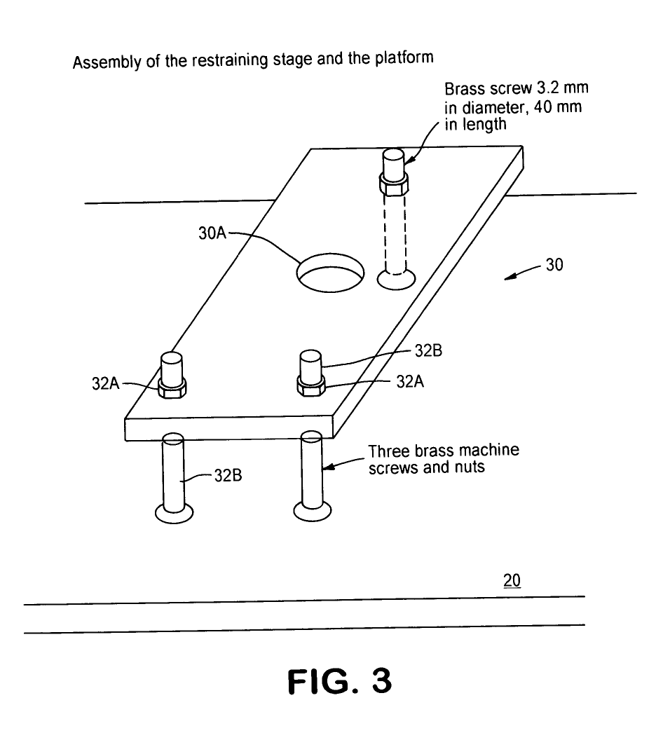

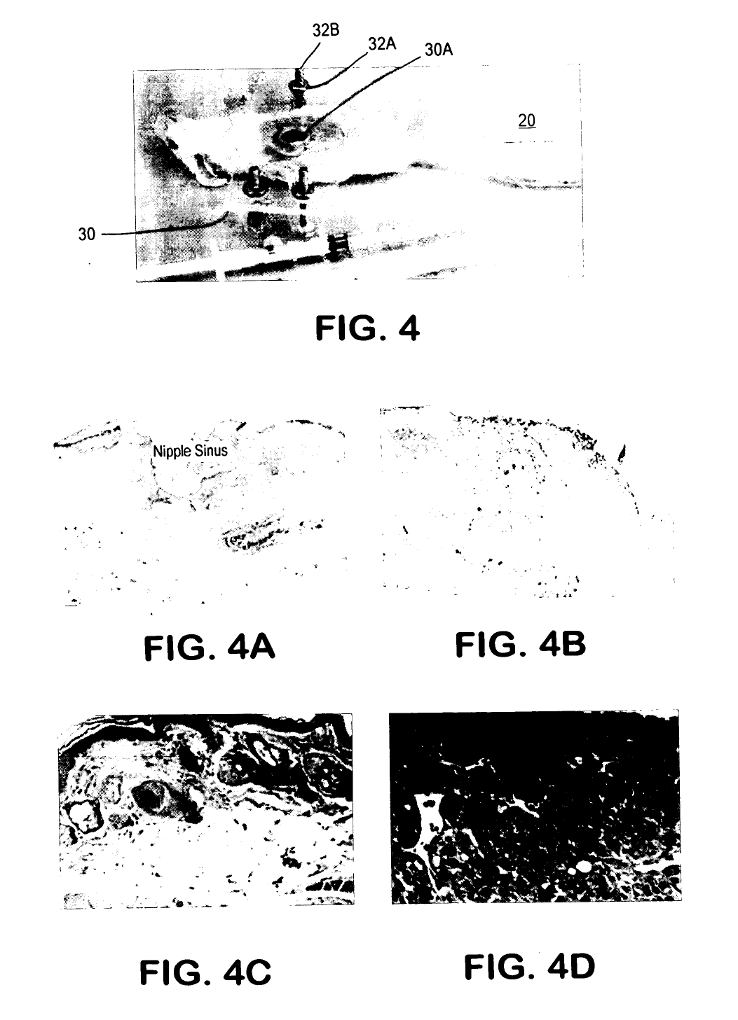

FIG. 2 depicts a microscopy stage, generally designated 20, upon which the rodent R is positioned and then secured by the restrainer plate, generally designated 30, which is mounted on top of the abdomen of rodent R with the central aperture 30A of the plate aligned with mammary window 10. Preferably restraining plate 30 is about 100 mm in length; 35 mm in width; and 5 mm in thickness. The central aperture 30A for being aligned with and receiving mammary window 10 is most suitably about 11-16 mm in diameter to facilitate alignment with mammary window 10 during intravital microscopy (see FIG. 4). The height of restraining plate 30 can be adjusted by turning three brass nuts 32A (see FIG. 3 and FIG. 4) of the three brass vertical screws 32B that secure restrainer plate 30 in spaced-apart relationship from microscopy stage 20. Preferably, restraining plate 30 is adjusted with brass nuts 32A of three brass vertical screws 32B so that slight pressure is applied on the rodent body R to reduce the respiratory movement during intravital microscopy. Most suitably both restraining plate 30 and microscopy stage 20 are formed from Plexiglass and mammary window 10 is an acrylic disk.

### Animals and Tumors Used in Method

Retired breeders or lactation weaned Fischer 344 rats averaged 210-230 g body weight and BALB/C mice weighted 20 to 25 g (available from Charles River Laboratories, Raleigh, N.C.) were used for rat and mouse mammary windows, respectively. All animals were housed in an animal facility with 12-h light-dark cycle and temperature control (24° C.) and access to bottled tap water and standard rodent chow ad libitum. All procedures and experiments were approved by the Duke Institutional Animal Care and Use Committee of Duke University in Durham, N.C.

Rat mammary adenocarcinoma R3230Ac cells or R3230Ac cells constitutively transfected with green fluorescence protein gene (GFP-R32330Ac) were used for orthotopic rat mammary window 10. Murine mammary carcinoma 4T1, or 4T1 cells constitutively transfected with GFP gene (GFP-4T1) were used for mouse mammary window 10. These GFP expressing tumor cell lines were derived as previously described in the art. Applicants discovered that the doubling times of GFP-transfected tumor cells in vitro are similar to parent non-transfected cell lines. There is no significant difference in growth rate between subcutaneously implanted GFP-transfected and non-transfected tumors (data not shown). Tumor cells were cultured in DMEM medium (available from Gibco Invitrogen Co., Grand Island, N.Y.) with 10% FBS (available from HyClone, Logan, Utah) and penicillin/streptomycin at 37° C. in air with 5% carbon dioxide before implantation.

### Anatomic Considerations

Rats and mice have six pairs of mammary glands, and they are referred to by location as cervical, cranial thoracic, caudal thoracic, abdominal, cranial inguinal, and caudal inguinal, or by numbers anterior to posterioras L1, R1, L2, R2, etc. The mammary glands are compound tubuloaveolar glands composed of a highly branched system of ducts and terminal secretary alveoli arranged in lobules. Each gland in the rat and mouse, has a single lactiferous duct entering the nipple. The duct widens to form the nipple sinus, which then opens onto the surface by way of the nipple canal. The nipple, nipple canal, and nipple sinus are lined by squamaous epithelium continuous with the epidermis. The second to fifth pairs of mamma on either side can be used for window surgery, but for convenience of intravital microscopy, mammary glands R3 or R4 were most commonly used by applicants.

### Surgical Procedures

The basic procedures in rats and mice are similar, except the window size. Animals were anesthetized with sodium pentobarbital (available from Abbott Laboratories, North Chicago, Ill.) given i.p. at 45 mg/kg body weight for rats and 80 mg/kg for mice. Animals were kept warm with a water blanket set at 37° C. The anterior aspect of lower thorax and abdomen was shaved and depilated with NAIR® (available from Carter-Wallace, Inc., New York, N.Y.). Skin was wiped twice with Chlorhexidine (available from Baxter-Healthcare, Co., Deerfield, Ill.) followed by alcohol. Surgery was performed with aseptic technique with the aid of a dissecting microscope. A circular incision with diameter of 8 mm for rats or 4 mm for mice was made on the skin around the nipple. The thin layer of skin around the base of the nipple was removed within the circular incision. The nipple was cut at its base and the nipple sinus was exposed. The lining epithelium was carefully removed.

Tumor cells or tumor fragments were then implanted into the nipple sinus. Acrylic disk 10 with multiple holes 10A (0.75 mm thick and 15 mm diameter for rats and 10 mm for mice) was placed into the wound with the tumor implant located at center. Disk 10 was sutured to the skin edge using modified subepidermal sutures. NEOSPORIN ointment was applied around the wound. To protect the window disk from being damaged by the animal after surgery, a lightweight rat Elizabethan collar (available from Harvard Apparatus, Holliston, Mass.) was applied before the animals emerged from anesthesia. For mice, an 18 mm wide adhesive bandage (Band-Aid available from Johnson & Johnson Medical Inc., Arlington, Tex.) was applied on the chest to protect the wound and window disk 10 for the first two days. Animals were kept in a cage with a wire floor to minimize wound contamination. The collars and bandages were removed two days after surgery.

### Tumor Implantation into Rodents

Different techniques were used for tumor implantation. For better monitoring of tumor cell morphological change and tumor-host interactions, single cell suspensions of GFP-expressing tumor cells (GFP-R4230Ac or GFP-4T1) were injected into the mammary tissues before window disk 10 was mounted. Tumor fragments derived from subcutaneously implanted tumors in a donor animal were transplanted into the nipple sinus in some studies, which yielded faster tumor growth.

For tumor cell injection, half-confluent cells were trysonized and washed with PBS twice immediately prior to surgery. Viable cell numbers were counted using trypan blue exclusion with a hemocytometer. Defined concentrations of viable cell suspensions were made by resuspending cell pellets with PBS. Cells were stored on ice prior to transplantation.

1) GFP-4T1 cell injection: 20μ of GFP-4T1 cells at 1×10⁵/ml in PBS was injected into the mouse mammary gland.

2) GELFOAM transplants. Since the brightness of GFP-R32330Ac cells in the mammary tissue after injection was not high enough to visualize cellular morphological change, a modified tumor cell transplantation was used. 3 μl of GFP-R32330Ac tumor cells at 1×10⁶/ml was soaked into a 1 mm³ piece of absorbable gelatin sponge (GELFOAM, available from Pharmacia & Upjohn, Kalamazoo, Mich.), which was placed into the nipple sinus.

3) Tumor fragments of R3230Ac. A 0.5 mm³ piece of tumor tissue from a donor animal with tumor growing subcutaneously on the thigh was placed into the nipple sinus for the first generation of mammary window 10, and fragments from orthotopically transplanted R3230Ac tumors in donor rats were used for later surgeries. Briefly, donor animals were anesthetized with NEMBUTAL (available from Abbott Laboratories, North Chicago, Ill.) at 50 mg/kg i.p. and tumor tissue was removed aseptically. After removal tumor tissue was rinsed with sterile saline, cut to 0.5 mm³ fragments in filtered DMEM, and maintained in DMEM on ice not longer than 2 hours before being implanted into the nipple sinus.

### Intravital Microscopy

Serial observations of window 10 were carried out on a CARL ZEISS MPS intravital microscope (available from Carl Zeiss, Hanover, Md.) starting from the day of tumor implantation (day 0). For microscopy at later time points, animals were anesthetized with the mixture of ketamine (available from Abbott Laboratories, North Chicago, Ill.) and xylazine (available from Bayer, Shawnee Mission, Kans.), i.p. (ketamine 45 mg/kg and xylazine 4 mg/kg for rats, double doses for mice). Rodents R were placed on a home-made Plexiglass stage 20 with an adjustable restrainer 30, and kept warm on a temperature controlled heating blanket (Homeothermic System, available from Harvard Apparatus) at 37° C. on microscope stage 20. Animals R were positioned to allow mammary window 10 to be aligned with hole 30A of restraining bar 30 (see FIG. 4). Visualization of tumor cells and microvessels in window 10 was similar to those for the dorsal skinfold window chamber which has been reported previously in the art, except white light epiillumination was used with a flexible fibroptic lamp (FIBER-LITE, Model 190, available from Dolan-Jenner Industries, Inc., Woburn, Mass.) instead of transillumination. Fluorescence epi-illumination was provided with a 100 W mercury-arc lamp (ATTOARC HBO, available from Carl Zeiss, Inc.). The microscope was equipped with a color video camera (CARL ZEISS ZVS-3C75DE) connected to a PC computer with a frame grabber and SCION image software (available from Scion Corporation, Frederick, Md.).

With the FITC filter (excitation 450-490 nm and emission 520 nm), GFP-expressing 4T1 cells were readily visualized, even at low magnificent (objective 5×). Cell morphology was recorded at higher magnification (objective ×2). For better visualization of blood vessels, rhodamine-dextran at a concentration of 10 mg/ml (40000 MW, available from Sigma, St. Louis, Mo.) 0.3 ml (rat) or 0.1 ml (mouse) was injected through a tail vein catheter. Rhodamine and FITC filters were switched alternatively to visualize tumor cells and the interrelationship between tumor cells and blood vessels. For identification of perfused vessels some images were video taped (SONY S-HVS video tape machine) for off-line study.

### Histology

Animals were sacrificed with EUTHASOL (available from Delmarva Laboratory, Inc., Midlothian, Va.) i.p. (0.3 ml for rats and 0.1 ml for mice), and the tumor with surrounding tissue were removed and fixed with 10% buffered formalin for one or two days then stored in 70% ethanol before processing and embedding. Five μm sections were stained with hemoxylin and eonosin.

### Testing Results and Discussion

### General Description

Both rats and mice R tolerated the procedure well. The main technical barrier was to prevent animals R from pulling off windows after surgery. Use of the rat Elizabethan collar and a chest bandage for mice for the first two postoperative days yielded a success rate of 85% for rats, and 80% for mice. Mammary window 10 could be maintained in place for up to 4 weeks without inflammation and with maintenance of good optics. Light scratches on the surface of window disks 10 did not interfere with intravital microscopy. Moderate body weight loss (about 5-8%) was observed for first two to three postoperative days in rats, when Elizabethan collars were applied. Daily face and eye cleaning was required during this period. No postoperative wound hemorrhage or infection occurred.

### Evolution of Tumor Fragment Implantation

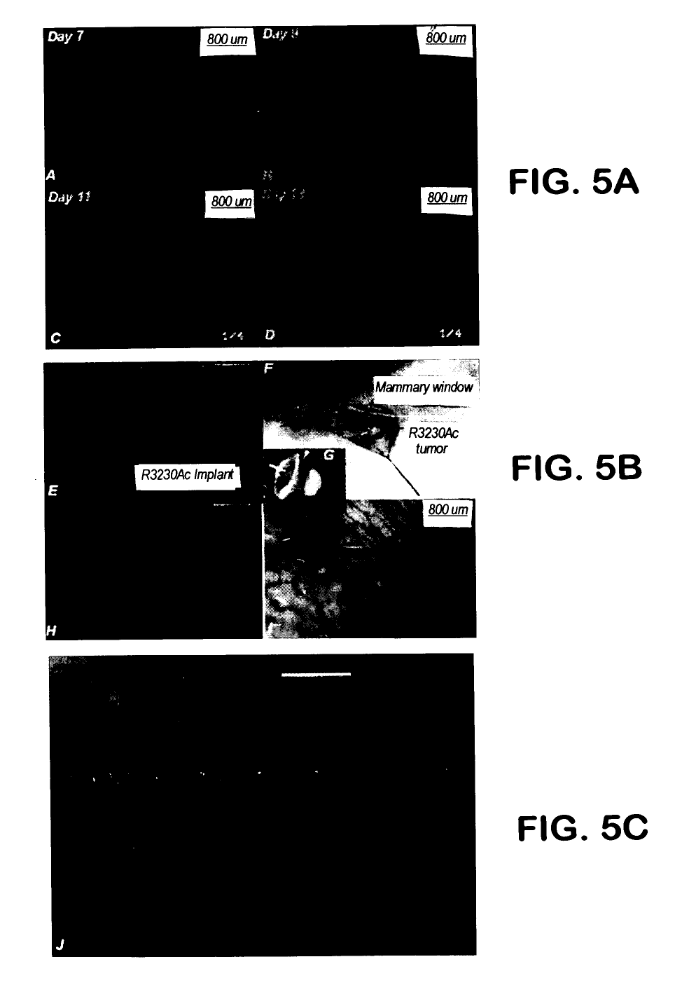

The histology of the nipple sinus of a mammary gland (R4) of a Fischer 344 rat and a BALB/C mouse are shown in FIG. 4A and FIG. 4C, respectively. R3230Ac tumor fragments implanted into the nipple sinus did not show any visible vessels during intravital microscopic observation within first three days, while surrounding areas of mammary gland tissue showed a hyperemic response, with increased vessel number, vasodilatation and turtuosity. Although no perfused vessels were seen during the first three days during intravital microscopy, histological tumor invasion into the epithelium of the nipple sinus was obvious on POD4 (FIG. 4B). Newly formed vessel sprouts initiated from preexisting vessels in surrounding host tissue were observed in the outer ring of the implant 3 days after implantation (FIGS. 5B, E and H). The sprouts extended into the implants and became perfused. Tumor size was stable during first five days and then began to grow as perfused vessels formed within the tumor parenchyma.

There was a mild inflammatory reaction in the tissue underneath window disks 10 within 3 to 4 days after surgery as a response to surgical intervention, manifested as mild edema and vasodilation in areas surrounding the mammary gland. However, once orthotopic tumors established, the tumor surface underneath acrylic window disk 10 showed only a thin layer of fibrin and collagen deposition without evidence of inflammation (FIG. 4D). Different from the subcutaneously implanted tumor in hind limb where the tumortends to be very cystic, the orthotopic R3230Ac tumors grew faster and remained solid even at sizes up to 2 cm in diameter (FIGS. 5B, F and G).

### Angiogenesis in GFP-R3230Ac Mammary Tumor

GFP-expressing tumor cell lines provide a valuable approach for serial observation of angiogenesis and tumor growth in this model, since GFP allows for direct demarcation of the exact location of tumor cells relative to blood vessels. The GFP also allows for clearer visualization of blood vessels as a result of fluorescent light absorption by hemoglobin. FIGS. 5A-5D represent selected time points of serial measurement of GFP-R32330Ac tumor growth and angiogenesis after GELFOAM implantation. Although the tumor cells express GFP, fluorescence was weak and tumor cells were not visible until POD 5. On POD 5 a few small faint fluorescent tumor clusters appeared in the outer zone of the GELFOAM implant near the border of surrounding host tissue. On POD 7, a bright green “ring” was apparent. The increase of GFP intensity in peripheral zone of implanted tumor probably resulted from active cell proliferation in these areas.

Active angiogenesis was observed from POD 5, manifested by appearance of numerous newly formed blood vessels, which were derived from vessels in surrounding host tissue and directed toward the inner area of implant. A unique pattern of vascularization observed in this model was microhemorrhagic appearing areas near the tips of the newly formed microvessels (FIGS. 4A-4D). Under higher magnification, most of these hemorrhagic spots were actually tiny blood-containing sprouts from new microvessels. These sprouts were replaced by perfused vessels on subsequent days resulting in the inward extension of new vessels. Further inside the implanted tumors, new sprouts appeared in the area where originally there were no visible vessels. These cyclic evolutions from vessel sprouts to new vessel formation continued until tumor vascularization was completed centrally.

As new vessels extended inward the implant, the green zone of tumor expanded inward, showing the close relationship between tumor growth and neovacularization.

### Morphological Behavior of GFP-4T1 Cells and Angiogenesis

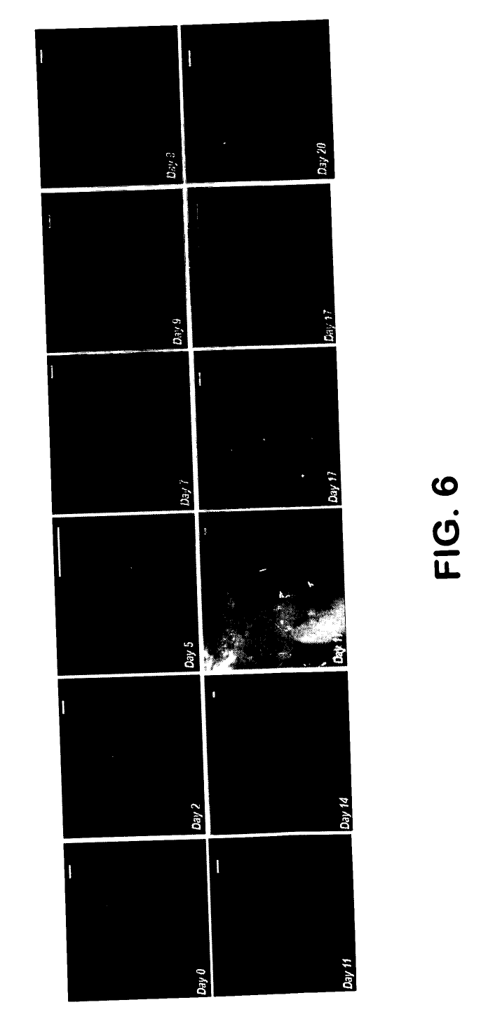

Tumor cells elongated and polarized toward pre-existing host vessels after inoculation. New vessel formation occurred at the stage where only a few hundred tumor cells presented (FIG. 5C). Most active angiogenesis always occurred in the peripheral zone of a growing tumor mass. Newly formed blood vessels originated from surrounding host tissue and grew inward toward the tumor central area. Similar to the findings in orthotopic GFP-R32330AC tumors on Fischer 344 rats, the consequent changes from blood-containing sprouts to perfused blood vessels, and inward vessel extension were clearly observed (FIG. 6).

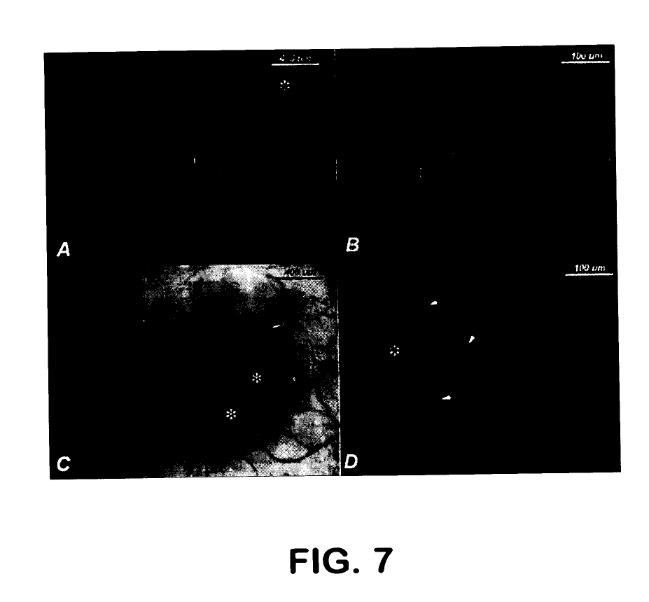

Microhemorrhagic appearing areas near the most interior angiogenic vessels was seen with both tumor types in FIG. 7. These regions consisted mainly of vessel sprouts derived from newly formed vessels admixed with microscopic hemorrhages devoid of apparent vascular walls. These vessel sprouts were replaced with microscopic hemorrhages devoid of apparent vascular walls. These vessel sprouts were replaced by newly formed vessels on subsequent days. The physiological implications of this unique pattern of neovascularization are unknown, but we have not observed it previously in either skin fold or brain window preparations. Such extravascular regions may be rich in plasma elements and blood cells. It is possible that circulating endothelial precursor cells (CEP) and multipotent adult progenitor cell (MAPC) from bone marrow may extravasate in these microhemorrhagic areas, which have been shown in recent studies to be of critical importance in tumor angiogenesis.

In all tumor lines applicants used for mammary windows 10, orthotopic breast tumors grew faster than the same type of subcutaneously grown tumor. Orthotopic 4T1 tumors also showed propensity to metastasize to regional lymph nodes and lungs (data not shown). This is not seen when these cells are transplanted into skin fold window chambers.

In the model of the present invention, applicants implanted tumor fragments or tumor cells in mammary tissue instead of the mammary fat pad, since it has been shown that epithelial-epithelial interactions, as well as stromal-epithelial interactions, are associated with the enhanced growth of mammary tumor cells transplanted into orthotopic sites. The nipple sinus provides a good anchoring place for implants and a good marker for centering the implant within mammary window 10.

In conclusion, the novel mammary window 10 of orthotopic breast cancer of rats and mice R provides a useful model to investigate the cellular behavior of implanted tumor cells in an orthotopic environment and to observe the earliest events during angiogenesis. The model should be quite suitable for studies that are typical for window chambers, such as study of tissue distribution of fluorescently labeled drugs, the host-tumor cell interactions, evaluation of the effects of various treatments and assessment of gene expression in situ, using stably transfected tumor cell lines containing fluorescent reporter genes.

### Alternate Embodiments

Although the primary application of the invention described hereinabove is for an orthotopic mammary window, the method and apparatus are broadly applicable to other sites where chronic physiologic observation of tissues is desired. For example, a window placed over the retroperitoneal space could be used for chronic observation of microvasculature on the surface of the kidney. The surgical implantation of the window for this particular application is described below.

Mice are anesthetized with NEMBUTAL at 100 mg/kg ip. The back is sterilized with EXIDIN/alcohol. A circular skin incision 6 mm in diameter is made over the left kidney Oust below the twelfth rib and left to the spine). The skin flap is removed and the dorsal muscles and fascia are dissected to expose the kidney. The window disk is inserted into the dissected circular incision on top of the kidney. The disk is secured with 4 to 5 subcutaneous sutures, similar to the method used in the mammary window described hereinabove. Animals tolerate the surgery well, and no additional measures are taken to protect the window since it located on the dorsum of the mouse trunk. This renal window can be used for studies on normal kidney function or for study of the orthotopically transplanted renal cancers.

The method could also be used to evaluate wound healing and tumor angiogenic responses to antiangiogenic drugs and other cytotoxic drugs. For this application, the window is implanted on the caudal back. For the “wound” model a collagen pellet is transplanted into the window and photographed through a stereomicroscope to follow vessel growth. For the tumor model 5×10\hat{ }6 HT29 colon carcinoma cells are transplanted under the window.

Additional sites for observation could include brain or even abdominal windows to facilitate chronic observation of the brain or liver. Such windows could also be implanted into other sites that are typically used for tumor transplantation, such as the subcutis of any portion of the body trunk or flank.

The apparatus and method of the present invention including the window disk, related elements and surgical techniques were originally discovered for orthotopic mammary tumors. Since the window disk is simple in construction with light weight, good optic quality, and is easy to be fixed to the skin over desired locations, it also may potentially be a universal tissue window for intravital microscopy on many orthotopic tumor models, such as soft tissue sarcomas (on the thigh, nape and back), lung cancer (thoracic window), colon, liver, stomach and renal cancers (abdominal window on proper locations), and as a cranial window for brain tumors.

It will be understood that various details of the invention may be changed without departing from the scope of the invention. Furthermore, the foregoing description is for the purpose of illustration only, and not for the purpose of limitation, as the invention is defined by the claims as set forth hereinafter.

What is claimed is:

1. A method for developing an orthotopic mammary carcinoma model for tumor microenvironment studies comprising the steps of:
(a) implanting tumor fragments or cells into the nipple sinus of an abdominal mammary gland of a rodent;
(b) surgically securing a mammary window in the mammary gland of the rodent and on top of the implanted tumor fragments or cells;
(c) periodically securing the rodent to a microscopy platform with a restrainer plate mounted on top of the abdomen of the rodent such that a central aperture in the restrainer plate is aligned with the mammary window; and
(d) observing selected aspects of tumor growth by intravital microscopy including microvascular function and angiogenesis in the orthotopic site.

2. The method according to claim 1 wherein the rodent is a rat or mouse.

3. The method according to claim 1 including implanting the tumor fragments or cells into the right thoracic and/or right abdominal mammary gland of the rodent.

4. The method according to claim 1 wherein surgically securing comprises securing a gas sterilized mammary window in place with aseptic surgical sutures.

5. The method according to claim 4 including surgically securing two or more mammary windows in two or more corresponding mammary glands of the rodent.

6. The method according to claim 4 including surgically securing a mammary window comprising an acrylic disk about 10.0-15.0 mm in diameter and about 0.75 mm in thickness.

7. The method according to claim 1 including leaving the surgically secured mammary window in place for up to 4 weeks on the rodent.

8. The method according to claim 1 including repeatedly securing the rodent to the microscopy platform with the restrainer plate to obtain repeated, continuous and non-invasive monitoring of tumor growth and angiogenesis.

9. The method according to claim 1 including observing by intravital microscopy with an intravital microscope having a color video camera connected to a PC adapted to perform frame grabbing.

10. The method according to claim 1 including observing by intravital microscopy including using white light epi-illumination provided by a flexible fiber optic lamp.

11. An orthotopic mammary carcinoma window for tumor microenvironment studies in rodents comprising:
(a) a transparent window for being sutured to the mammary gland of a rodent on top of tumor fragments or cells that have been implanted in the nipple sinus;
(b) a microscopy platform;
(c) a restrainer plate mounted to said microscopy platform and having a top plate with an aperture therein for alignment with said transparent window and said aperture being of a larger diameter than said window; and
(d) an adjustment mechanism to vertical adjust said retainer plate relative to said microscopy platform so as to provide a space therebetween for restraining the rodent to allow for intravital microscopy through said window to observe tumor growth.

12. The window according to claim 11 wherein said window is acrylic and has a diameter between about 10.0 and 15.0 mm and a thickness of about 0.75 mm.

13. The window according to claim 11 wherein said microscopy platform is a planar sheet of plexiglass.

14. The window according to claim 11 wherein said aperture within said restrainer plate has a diameter between about 11.0 and about 16.00 mm.

15. The window according to claim 11 wherein said adjustment mechanism comprises a plurality of adjustment screws extending between said microscopy platform and said restrainer plate.

16. A method for developing an orthotopic carcinoma model for tumor microenvironment studies comprising the steps of:
(a) implanting tumor fragments or cells into a site of interest of a rodent;
(b) surgically securing a window in the site of interest of the rodent and on top of the implanted tumor fragments or cells;
(c) periodically securing the rodent to a microscopy platform with a restrainer plate mounted on the rodent such that a central aperture in the restrainer plate is aligned with the window; and
(d) observing selected aspects of tumor growth by intravital microscopy including microvascular function and angiogenesis in the orthotopic site.

17. The method according to claim 16 wherein the rodent is a rat or mouse.

18. The method according to claim 16 including implanting the tumor fragments or cells into a site selected from the group comprising: the kidney of the rodent; the brain of the rodent; the liver of the rodent; the lung of the rodent; the colon of the rodent; and the stomach of the rodent.

19. The method according to claim 16 wherein surgically securing comprises securing a gas sterilized mammary window in place with aseptic surgical sutures.

20. The method according to claim 19 including surgically securing a mammary window comprising an acrylic disk about 10.0-15.0 mm in diameter and about 0.75 mm in thickness.

21. The method according to claim 16 including repeatedly securing the rodent to the microscopy platform with the restrainer plate to obtain repeated, continuous and non-invasive monitoring of tumor growth and angiogenesis.

22. The method according to claim 16 including observing by intravital microscopy with an intravital microscope having a color video camera connected to a PC adapted to perform frame grabbing.

23. The method according to claim 16 including observing by intravital microscopy including using white light epi-illumination provided by a flexible fiber optic lamp.

24. An orthotopic carcinoma window for tumor microenvironment studies in rodents comprising:
(a) a transparent window for being sutured to a site of interest of a rodent on top of tumor fragments or cells that have been implanted in the site of interest;
(b) a microscopy platform;
(c) a restrainer plate mounted to said microscopy platform and having a top plate with an aperture therein for alignment with said transparent window and said aperture being of a larger diameter than said window; and
(d) an adjustment mechanism to vertical adjust said retainer plate relative to said microscopy platform so as to provide a space therebetween for restraining the rodent to allow for intravital microscopy through said window to observe tumor growth.

25. The window according to claim 24 wherein said window is acrylic and has a diameter between about 10.0 and 15.0 mm and a thickness of about 0.75 mm.

26. The window according to claim 24 wherein said microscopy platform is a planar sheet of plexiglass.

27. The window according to claim 24 wherein said aperture within said restrainer plate has a diameter between about 11.0 and about 16.00 mm.

28. The window according to claim 24 wherein said adjustment mechanism comprises a plurality of adjustment screws extending between said microscopy platform and said restrainer plate.
